(19) United States
(12) Patent Application Publication
(10) Pub. No.: US 2020/0323791 A1
(43) Pub. Date: Oct. 15, 2020

(54) CANNABIS DELIVERY WITH PROTECTIVE GLAZE COATING

(71) Applicant: Mantrose-Haueser Co., Inc., Westport, CT (US)

(72) Inventors: Stephen A. Santos, Cumberland, RI (US); William E. Barrie, Fairfield, CT (US); Karen M. Murphy, Summerville, SC (US)

(21) Appl. No.: 16/839,380

(22) Filed: Apr. 3, 2020

Related U.S. Application Data
(60) Provisional application No. 62/832,532, filed on Apr. 11, 2019.

(51) Int. Cl. A61K 31/05; A61K 9/00; A61K 9/70; A61K 31/352

(52) U.S. Cl. CPC A61K 31/05; A61K 31/352; A61K 9/7015; A61K 9/0056

(57) ABSTRACT

A cannabis-containing edible product comprises an edible substrate and a predetermined amount of a protective glaze coating on the edible substrate. The protective glaze coating comprises an edible, film-forming resin which has been applied by liquid coating techniques and contains cannabis in a predetermined concentration such that the cannabis-containing edible product contains a predetermined amount of cannabis.

### CROSS-REFERENCE TO RELATED APPLICATION

This application claims priority to and all benefit of U.S. Provisional Patent Application Ser. No. 62/832,532, filed on Apr. 11, 2019 and entitled CANNABIS DELIVERY WITH PROTECTIVE GLAZE COATING, the entire disclosure of which is incorporated herein by reference.

### BACKGROUND AND SUMMARY

US 2012/046351 to Hospodor describes a method for accurately controlling the amount of medicinal cannabis consumed by a user by including the cannabis in an extract or food topping which is inserted into or applied onto a foodstuff.

Protective glaze coatings made from shellac or analog, when applied to various foodstuffs and other materials, are not only especially effective in connection with accurately controlling the amount of cannabis consumed, but also effectively protect the applied cannabis against loss and/or degradation during storage, handling and use.

Thus, this invention provides a cannabis-containing edible product comprising an edible substrate and a predetermined amount of a protective glaze coating on at least a portion of the surface of the edible substrate, wherein the protective glaze coating contains cannabis in a predetermined concentration such that the cannabis-containing edible product contains a predetermined amount of cannabis, wherein the protective glaze coating is essentially tasteless and comprises an edible, film-forming resin.

In addition, this invention also provides a process for producing a cannabis-containing edible product which contains a predetermined amount of cannabis, the process comprising applying a predetermined amount of a liquid glaze coating composition onto at least a portion of the surface of the edible substrate followed by drying to produce a protective glaze coating, the liquid glaze coating composition comprising an edible, film-forming resin, a carrier liquid and cannabis in a predetermined concentration such that the cannabis-containing edible product contains a predetermined amount of cannabis, wherein the liquid glaze coating composition is formulated so that the protective glaze coating obtained is essentially tasteless and comprises an essentially continuous layer of an edible, film-forming resin.

### DETAILED DESCRIPTION

### Cannabis

This invention relates to an improved method for accurately dosing the amount of cannabis consumed by a user.

The two main varieties of the cannabis sativa plant are marijuana and hemp. Both contain significant quantities of the psychoactive compound tetrahydrocannabinol (THC) and the non-psychoactive compound cannabidiol (CBD), although marijuana contains a much higher proportion of THC than hemp. However, both contain more than 500 compounds, among them at least 113 cannabinoids which are a class of psychoactive chemical compounds which act on the cannabinoid receptors in cells that alter neurotransmitter release in the brain.

Additional compounds of interest in the cannabis sativa plant are tetrahydrocannabinol carboxylic acid (THC-COOH), cannabidiolic acid (CBDA), cannabigerolic acid (CBGA), cannabinol (CBN), cannabigerol (CBG) and cannabidvarin (CBDV). Cannabinoids as a general class of compounds are also of interest, as are non-psychoactive compounds in the cannabis sativa plant which exhibit some other type of pharmacological activity such as ant-inflammatory, immunosuppressive, analgesic, anxiolytic and/or anti-cancer effects. Still another class of compounds of interest in the cannabis sativa plant are those which exhibit essentially no pharmacological activity such as a variety of different terpenes, for example.

Cannabis has been consumed by humans for centuries. Most commonly this has been done by smoking raw leaves of the cannabis sativa plant and/or by eating foodstuffs containing these raw leaves. Extracts have also been used to supply cannabis for consumption by eating, smoking, inhaling, etc.

In this regard, it is common practice to recover the active ingredients found in the cannabis sativa plant by some sort of extraction process in which leaves, stems or other portions of the plant, usually ground or crushed, are contacted with a suitable liquid extractant. Although ethanol and isopropanol are commonly used for this purpose, other compounds which are liquid at standard temperature and pressure (STP) such as water, other C₁-C₅ oxygenated organic solvents, etc., can also be used. In addition, liquefied gases such as propane, butane and carbon dioxide can also be used under elevated pressure.

Depending on the particular liquid extractant used and the particular cannabis ingredient involved, the cannabis ingredient may be dissolved in the liquid extractant in the sense of forming a true (i.e., molecular) solution, or it may be dispersed or emulsified in the liquid extractant in the case of solid or liquid cannabis ingredients, respectively. Or the liquid extractant may be totally absent such as occurs, for example, when liquefied gases used for extraction under elevated pressure are allowed to evaporate at atmospheric pressure.

These extracts can be processed in many different ways such as, for example, by evaporating off the liquid extractant to produce a concentrate, by purifying to recover pharmacologically active ingredients from inactive ingredients, by separating different pharmacologically active ingredients from one another such as, for example, by separating psychoactive compounds such as THC from non-psychoactive compounds such as CBD, and so forth.

As the potential pharmacological benefits of cannabis and its ingredients have become more apparent, much effort has been undertaken to isolate, study and synthetically reproduce individual cannabinoids and other compounds found in the cannabis sativa plant, including mixtures thereof, as well as analogs of such compounds and mixtures. In this context, an “analog” of a compound or mixture of compounds found in the cannabis sativa plant will be understood to mean a compound or mixture which exhibits a physiological effect on humans which is essentially the same as that exhibited by the compound or mixture derived from this plant, even if more or less intense.

Much effort has also been undertaken to develop methods for purifying and concentrating extracts of compounds and mixtures of compounds derived from the cannabis sativa plant such as shown, for example, in U.S. Pat. Nos. 7,724,881 and 7,592,468. Another example of such a concentrated extract is 99+% pure CBD isolate powder.

The purpose of this invention is to provide an improved method for accurately controlling the amount or dose of cannabis consumed by a user regardless of the form this cannabis is in (e.g., ground or crushed leaves, extract, individual compounds, mixtures of such compounds, physical and/or chemical agglomerations of such compounds, reaction products of such compounds, or some other combination of such compounds), regardless of the identity of the particular cannabis ingredient used, regardless of the physiological effect provided by the cannabis ingredient, if any, regardless of whether the cannabis ingredient is naturally derived or synthetically produced and regardless of whether the cannabis ingredient has been purified, concentrated or remains in an essentially raw condition.

Thus, it will be understood that “cannabis” and “cannabis ingredient” as used in this disclosure broadly refer to any and all such products derived from the cannabis sativa plant, including synthetic counterparts, regardless of form, identity, chemical composition, physiological efficacy, purity and/or concentration. In addition, it should also be understood that, when the source of the cannabis ingredient used in this invention is an extract which also contains a liquid extractant, “cannabis” and “cannabis ingredient” refer only to the extracted materials whether they are dissolved, dispersed or emulsified in the liquid extractant. They do not refer to or include the liquid extractant that might be present, if any. Similarly, when the source of the cannabis ingredient used in this invention is a synthetic version of any such extract, “cannabis” and “cannabis ingredient” refer only to the synthetic analog of the extracted materials. They do not refer to or include any carrier liquid that might be present.

### Edible Substrate

In accordance with this invention, cannabis is supplied to a user in a precisely controlled amount by including the cannabis in a protective glaze coating which is applied to an edible substrate to be eaten by the user.

For this purpose, essentially any edible product can be used as the edible substrate. For example, tablets and pills can be used, as can various different types of “hard” food products such as apples and pears, dried fruits such as raisins and prunes, nuts, hard candies such as gumballs, malt balls, sugar candies, and hard chocolates (e.g., both regular and peanut M&M's). Food products with a “medium hardness” can also be used, examples of which include softer candies such as gum drops, gummies and softer chocolates. Fragile foodstuffs such as crackers, potato chips and confections can also be used.

In addition, “soft” food products can also be used, examples of which include baked goods such as cookies, muffins, donuts, bagels, pies, cakes, etc. Including cannabis in the protective glaze coating of this invention rather than incorporating it into the dough used to make of the baked good has the additional advantage of avoiding heating the cannabis to temperatures high enough to cause it to vaporize and/or decompose during the baking process.

### Liquid Glaze Coating Composition—Carrier Liquid

The protective glaze coating of this invention is formed by liquid coating techniques in which a suitable liquid glaze coating composition containing an edible, film-forming resin dissolved and/or dispersed in a suitable carrier liquid is applied to a substrate and then dried.

In some embodiments, this carrier liquid will be water. In other embodiments, this carrier liquid will be an organic solvent such as, for example, a C₁-C₅ oxygen-containing organic solvent such as ethanol, acetone, isopropanol, methanol, ethylene glycol, glycerol and mixtures thereof. C₁-C₃ alcohols containing 1-3 hydroxyl groups such as methanol, ethanol, isopropanol, butanol, ethylene glycol, glycerol and ethyl acetate are desirable. Food grade organic solvents of this type, especially food grade alcohols, are especially desirable. In this context, “food grade” will be understood to refer to ingredients which are Generally Recognized As Safe (“GRAS”) under sections 201(s) and 409 of the Federal Food, Drug, and Cosmetic Act (the Act). Ethanol and isopropanol are especially preferred. Also, when ethanol is used, it may include a suitable denaturant so as to make it qualify as a “denatured spirit.” See, 27 CFR 21.151.

In still other embodiments, the carrier liquid can be a mixture of organic solvent and water. If so, the organic solvent is preferably miscible with water. When mixtures of organic solvent and water are used, the weight ratio of organic solvent to water can vary widely, and essentially any amount can be used. For example, the weight ratio of organic solvent to water can be ≥0.1:1, ≥0.2:1, ≥0.3:1, ≥0.4:1, ≥0.5:1, ≥0.6:1, ≥0.7:1, ≥0.8:1, ≥0.9:1 or even ≥0.95:1. In addition, it can also be ≤0.9:1, ≤0.8:1, ≤0.7:1, ≤0.6:1, ≤0.5:1, ≤0.4:1, ≤0.3:1, ≤0.2:1, ≤0.1:1 or even ≤0.05:1.

In those embodiments in which the carrier liquid is an organic solvent and the edible, film-forming resin is dissolved in this carrier liquid, if any water is present, the weight ratio of the water to the organic solvent will generally be ≤1:1, more typically ≤0.5:1, ≤0.4:1, ≤0.3:1, ≤0.2:1, ≤0.1:1, ≤0.05:1, ≤0.01:1, or even ≤0.005:1.

The concentration of the edible, film-forming resin in the liquid glaze coating composition of this invention can vary widely and essentially any concentration can be used. For example, this concentration can be as little as 1 wt. % and as high as 50 wt. % or higher, based on the weight of the liquid glaze coating composition as a whole. Normally, however, this concentration can be ≥2 wt. %, ≥3 wt. %, ≥5 wt. %, ≥7.5 wt. %, ≥10 wt. %, ≥15 wt. %, ≥20 wt. %, ≥25 wt. %, ≥30 wt. %, ≥35 wt. %, or even ≥40 wt. %, on this same basis. In addition, this concentration can be ≤45 wt. %, ≤40 wt. %, ≤30 wt. %, ≤25 wt. %, ≤20 wt. %, ≤17.5 wt. %, or even ≤15 wt. % on the same basis.

The edible, film-forming resin can be dissolved in the carrier liquid, dispersed in the carrier liquid or both, which depends primarily on the particular film-forming resin or resins used as well as the particular carrier liquid or liquids used. In this regard, as previously indicated, the technology for making tough, hard, durable, strongly adherent, edible, thin protective coatings on various edible substrates using liquid coating techniques is highly developed. That being the case, for each edible, film-forming resin used, those skilled in the art should have no difficulty in selecting the particular carrier liquid to use, the concentration of this edible, film-forming resin in this carrier liquid and whether this resin is dissolved and/or dispersed in this carrier liquid in order to produce the protective glaze coatings of this invention.

### Protective Glaze Coating—Edible Film-Forming Resin

The protective glaze coating of this invention is formed from an edible, film-forming resin which is capable of forming thin protective film coatings when applied by liquid coating techniques.

In one embodiment, this edible film-forming resin is ethanol-soluble.

Examples of suitable ethanol-soluble, edible, film-forming resins that can be used for this purpose include shellac, zein, ethyl cellulose, and certain grades of hydroxypropyl cellulose. Mixtures of these ethanol-soluble, edible, film-forming resins can also be used. Shellac, zein and mixtures thereof are preferred. Bleached shellac, especially refined (i.e., dewaxed) bleached shellac, as well as dewaxed orange shellac, are particularly useful for this purpose.

Shellac is a naturally occurring thermoplastic obtained from secretions of the female lac bug. It exhibits a remarkable combination of properties including low permeabilities to oxygen, water vapor, CO2, ethylene and various odors, low lipid solubility, excellent color and excellent clarity.

Shellac is obtained from seedlac, an insect secretion, by removing debris from the seedlac and then further processing the seedlac to obtain the desired product. Commercially, shellac is available in two different types, bleached shellac and orange shellac. Moreover, both of these shellacs are available in refined (i.e., dewaxed) as well as unrefined (regular) versions. In addition, each of these four different varieties of shellac are available in different physical forms, e.g., solid flakes and aqueous and/or alcohol solutions. In addition, some of these different varieties are also available in different grades. For example, dewaxed orange shellac is available in a variety of different grades ranging from faint orange to intense orangish red.

As described in U.S. Pat. No. 6,348,217, bleached shellac is made by dissolving seedlac in aqueous alkali and then adding a bleaching agent such as sodium hypochlorite. The product so obtained is then precipitated and dried to produce regular bleached shellac. Alternatively, the dissolved bleached shellac can be refined by known techniques to remove its wax content before precipitating and drying, thereby producing dewaxed bleached shellac. In contrast, regular orange shellac is made by melting seedlac, sieving out the insolubles and then solidifying and flaking the product so obtained. Meanwhile, dewaxed orange shellac is made by dissolving the seedlac in alcohol, straining out the insolubles, filtering out wax particles and passing the solution so obtained through activated carbon to decolorize before solidifying and flaking. In accordance with this invention, each of these different types of shellac can be used to make the protective glaze coatings of this invention.

Meanwhile, zein is a class of prolamine proteins found in maize (corn). Pure zein is clear, odorless, tasteless, hard, water-insoluble, ethanol-soluble and edible. It is usually manufactured as a powder from corn gluten meal and has a variety of different uses including coatings for candy, nuts, fruit, pills, other encapsulated foods and drugs, paper cups, soda bottle cap linings, clothing fabrics and the like. For a fuller description of zein, please see Lawton, Zein: A History of Processing and Use, Cereal Chem. Vol. 79, No. 1, ppl-18, 2002.

Ethyl cellulose is a derivative of cellulose in which some of the hydroxyl groups on the repeating glucose units are converted into ethyl ether groups. It is also colorless, odorless, tasteless, hard, water-insoluble, ethanol-soluble and edible. It is widely available commercially and mainly used as a thin-film coating material for coating paper, vitamin and medical pills, and for thickeners in cosmetics and in industrial processes. See, for example, ETHOCELL Ethyl Cellulose A Technical Review, Technical Bulletin, 02-2016, Dow Chemical Company.

Another class of edible, film-forming resins that can be used to make the protective glaze coatings of this invention are those which are water-soluble. Generally speaking, these water-soluble resins can be broken down into the following categories: (1) hydrocolloids, (2) polypeptides, (3) lipids, (4) cellulose derivatives and alkali soluble resins such as shellac.

Specific examples include starches, gum arabic, xantham gum, other polysaccharides such as alginates, carrageenan, chitosan and pectin, alkali soluble shellac, edible proteins such as wheat gluten, soy, casein, whey, peanut proteins, fish proteins and mung bean proteins, and cellulose derivatives such as methyl cellulose, carboxymethylcellulose, certain grades of hydroxypropyl cellulose and hydroxypropyl methylcellulose. Mixtures of these edible, water-soluble, film forming resins can also be used.

Yet another class of edible, film-forming resins that can be used to make the protective glaze coatings of this invention are those which are soluble in mixtures of ethanol and water. An example of this type of resin is hydroxypropyl cellulose (“HPC”), which is an ether of cellulose in which some of the hydroxyl groups in the repeating glucose units have been hydroxypropylated, normally using propylene oxide. Depending on the extent of hydroxypropylation, molecular weight and other factors, some grades of HPC are soluble in ethanol, other grades are soluble in water, while still others are soluble in mixtures of ethanol and water.

In addition to these resins, mixtures of these resin can also be used. That is to say mixtures of one or more ethanol-soluble resins, one or more water-soluble resins, and/or one or more resins which are soluble in ethanol/water mixtures can be used. If so, the weight ratio of these different types of edible, film-forming resins to one another can vary widely and essentially any weight ratio can be used. For example, the weight ratio of water-soluble to ethanol-soluble resins can be ≥0.1:1, ≥0.2:1, ≥0.3:1, ≥0.4:1, ≥0.5:1, ≥0.6:1, ≥0.7:1, ≥0.8:1, ≥0.9:1 or even ≥0.95:1. In addition, it can also be ≤0.9:1, ≤0.8:1, ≤0.7:1, ≤0.6:1, ≤0.5:1, ≤0.4:1, ≤0.3:1, ≤0.2:1, ≤0.1:1 or even ≤0.05:1.

However, in those embodiments of the invention in which the protective glaze coating is formed from an ethanol-soluble resin which has been dissolved in an organic solvent, if the liquid glaze coating composition also contains a water-soluble resin, the weight ratio of water-soluble to ethanol-soluble resin will generally be ≤0.5:1, ≤0.4:1, ≤0.3:1, ≤0.2:1, ≤0.1:1 or even ≤0.05:1. Such protective coatings, i.e., protective coatings based on an ethanol-soluble resin dissolved in an organic solvent such as ethanol, can be entirely free of any added water-soluble, film-forming resin, if desired.

### Protective Glaze Coating—Cannabis Content

In accordance with this invention, precisely controlled amounts of cannabis are supplied to a user by coating various different edible substrates with a predetermined (i.e., controlled) amount of a protective glaze coating and, in addition, by including a predetermined (i.e., controlled) concentration of a cannabis ingredient in the protective glaze coating. The amount of cannabis supplied to the user is precisely controlled by this approach, not only because the amount of cannabis incorporated into the edible substrate can be precisely metered but also because loss and/or degradation of the cannabis ingredient over time which could otherwise occur when the inventive cannabis-containing edible product is stored, packaged and shipped is largely eliminated by the protective glaze coating.

The cannabis ingredient used for this purpose can be in any form. For example, it can be in the form of ground or crumbled leaves, stems or other portions of the cannabis sativa plant, in which case it will normally be dispersed in the carrier liquid of the liquid glaze coating composition.

Alternatively, if the cannabis ingredient is derived from an extract of the cannabis sativa plant or is produced synthetically, this cannabis ingredient can be dissolved, dispersed and/or emulsified in the carrier liquid of the liquid glaze coating composition depending on a variety of factors including the particular carrier liquid, cannabis ingredient and film-forming resin used, the concentrations of these ingredients, whether the cannabis ingredient has been concentrated and/or purified, whether the cannabis ingredient has been produced synthetically, and whether any physical and/or chemical agglomeration or other reaction has occurred, etc.

For example, when the cannabis ingredient comprises an ethanol-soluble compound or mixture of compounds and the organic solvent comprises a C₁-C₃ alcohol containing 1-3 hydroxyl groups, the cannabis ingredient may be dissolved in this organic solvent in the sense of forming a true (molecular) solution. On the other hand, if water is used as the carrier liquid, the same ethanol-soluble cannabis compounds could be emulsified and/or dispersed in the carrier liquid instead.

It is also contemplated that the cannabis ingredient can be incorporated into an edible, film-forming resin which is dispersed in the carrier liquid.

The concentration of the cannabis ingredient in the liquid glaze coating composition of this invention can vary widely, and essentially any concentration can be used. Among other things, this concentration will depend on the particular form of the cannabis ingredient used (e.g., ground or crushed leaves, extract, concentrated extract, purified extract, individual compounds, mixtures of such compounds, etc.), the particular cannabis compound or mixture of compounds being used, the nature and concentration of the edible film-forming resin in the composition, the desired amount of protective glaze coating to be formed and the desired concentration of cannabis ingredient in this protective glaze coating.

Within these broad considerations, the concentration of the cannabis ingredient can be as little as 1 wt. % and as high as 50 wt. % or higher, based on the weight of the liquid glaze liquid coating composition as a whole. However, the concentration of the cannabis ingredient can also be ≥2 wt. %, ≥3 wt. %, ≥5 wt. %, ≥7.5 wt. %, ≥10 wt. %, ≥15 wt. %, ≥20 wt. %, ≥25 wt. %, ≥30 wt. %, ≥35 wt. %, or even ≥40 wt. %, on this same basis. In addition, this concentration can also be ≤45 wt. %, ≤40 wt. %, ≤30 wt. %, ≤25 wt. %, ≤20 wt. %, ≤17.5 wt. %, or even ≤15 wt. % on the same basis.

With respect to the relative amounts of ingredients in the liquid glaze coating composition of this invention and hence the protective glaze coating which it forms, the weight ratio of cannabis ingredient to edible, film forming resin or resins can also vary widely and can be as little as 0.005:1 to as much as 1:1. For example, this ratio can be ≥0.01:1, ≥0.02:1, ≥0.05:1, ≥0.075:1, ≥0.1:1, ≥0.15:1, ≥0.2:1, ≥0.25:1, ≥0.3:1, ≥0.35:1, ≥0.4:1, ≥0.5:1, ≥0.6:1, ≥0.7:1, ≥0.8:1, or even ≥0.9:1, depending on the particular cannabis ingredient used as well as the particular edible, film-forming resin used. Similarly, this ratio can be ≤0.9:1, ≤0.8:1, ≤0.7:1, ≤0.6:1, ≤0.5:1, ≤0.45:1, ≤0.4:1, ≤0.35:1, ≤0.3:1, ≤0.35:1, ≤0.2:1, ≤0.15:1, or even ≤0.1:1, also depending on the particular cannabis ingredient and edible, film-forming resin used.

When the cannabis ingredient used is a raw product such as ground or crushed leaves or stems, the weight ratio of this cannabis ingredient to the edible, film forming resin will normally be towards the upper ends of the above ranges, e.g., ≥0.15:1, ≥0.2:1, ≥0.25:1, ≥0.3:1, ≥0.35:1, ≥0.4:1. ≥0.45:1≥0.5:1, ≥0.6:1, ≥0.7:1, ≥0.8:1, or even ≥0.9:1 In contrast, when the cannabis ingredient is a particular compound or mixture of compounds such as THC and/or CBD, the weight ratio of this cannabis ingredient to the film forming resin will normally be towards the lower ends of the above ranges, e.g., ≤0.7:1, ≤0.6:1, ≤0.5:1, ≤0.4:1, ≤0.35:1, ≤0.3:1, ≤0.25:1, ≤0.2:1, ≤0.15:1, or even ≤0.1:1.

As previously indicated, the two most prevalent compounds found in cannabis are the psychoactive compound tetrahydrocannabinol (THC) and the non-psychoactive compound cannabidiol (CBD). When a cannabis-containing edible product of this invention is specifically designed to provide one or both of these compounds, the concentration of this compound, or the mixture of both of these compounds, can be the same as indicated above.

For example, the combined concentration of these compounds can be ≥0.5 wt. %, ≥1 wt. %, ≥1.5 wt. %, ≥2 wt. %, ≥3 wt. %, ≥5 wt. %, ≥7.5 wt. %, or even ≥10 wt. %, ≥15 wt. %, ≥20 wt. %, ≥25 wt. %, ≥30 wt. %, ≥35 wt. %, or even ≥40 wt. %, and ≤45 wt. %, ≤40 wt. %, ≤35 wt. %, ≤30 wt. %, ≤25 wt. %, ≤20 wt. %, ≤17.5 wt. %, ≤15 wt. %, or even ≤12.5 wt. % on the same basis as indicated above. Similarly, the weight ratio of both of these compounds together with respect to the edible film-forming resin can be ≥0.002:1, ≥0.005:1, ≥0.01:1, ≥0.02:1, ≥0.05:1, ≥0.075:1, ≥0.1:1, ≥0.15:1, ≥0.2:1, ≥0.3:1, ≥0.4:1, ≥0.5:1, ≥0.6:1, ≥0.7:1, ≥0.8:1, or even ≥0.85:1 and ≤0.8:1, ≤0.7:1, ≤0.6:1, ≤0.5:1, ≤0.45:1, ≤0.4:1, ≤0.35:1, ≤0.3:1, ≤0.25:1, ≤0.2:1, or even ≤0.15:1.

Finally, as indicated above, for the purposes of this disclosure any liquid extractant or other carrier liquid which might be present in an extract or other cannabis source is not regarded as part of the “cannabis” or “cannabis ingredient of this invention, even if present in the liquid glaze coating composition ultimately produced. It will therefore be appreciated that the above concentrations and weight ratios refer only to the cannabis ingredient itself and do not include any such liquid extractant or carrier liquid that might be associated with this cannabis ingredient.

### Optional Ingredients

In addition to the ingredients discussed above, the liquid glaze coating compositions of this invention can contain additional ingredients for improving the performance and/or reducing the cost of the protective glaze coatings they provide. Examples include dispersing agents, plasticizers, denaturants for alcohols, crosslinking agents, detackifiers, surfactants, preservatives, and other ingredients which improve the barrier properties of the protective glaze coating which are ultimately formed. If so, the total amount of these ingredients is normally very small, e.g., ≤10 wt. %, ≤5 wt. %, ≤3 wt. %, ≤2 wt. %, or even ≤1 wt. %, based on the weight of the composition as whole.

### Method of Application

As indicated above, the protective glaze coatings of this invention are formed by liquid coating techniques in which a thin, essentially continuous, resin film coating is produced by evaporation of a carrier liquid.

As well-known in the art, liquid coating techniques of this type are capable of producing protective resin film coatings on a wide variety of different substrates which coatings not only are very thin (e.g., ≤50 micron in dry thickness) but also have a controlled and uniform thickness as well as being essentially continuous, tough, hard, durable, and strongly adherent to their substrates. In this context, “dry thickness” and “dry coating thickness” will be understood to mean the thickness of a coating which is formed by liquid coating techniques after the carrier liquid in the coating composition used to form the coating has evaporated. In addition, “essentially continuous” will be understood to mean that the protective glaze coating of this invention is like conventional pharmaceutical glazes and confectioners glazes in terms of the continuity of the film coating that is formed. In other words, in the same way as these conventional resin film coatings, the edible, film-forming resin of the inventive protective glaze coating is not merely dispersed or distributed in the coating as a discrete, discontinuous ingredient but rather forms a continuous resin film which covers the entire surface area to which the coating is applied, with any pinholes or other minor imperfections that may be present in this film, if any, being insignificant.

Liquid coating techniques are widely used to provide colorless, odorless, tasteless, hard, tough, edible, thin, edible, gloss-enhancing protective coatings on a wide variety of different edible substrates including tablets, pills and various food products. In industry, these protective resin film coatings when made from shellac are normally referred to as “pharmaceutical glazes” or “confectioner's glazes,” depending on the particular substrate being coated. When made from other resins, these protective film coatings are normally referred to simply as “film coatings.” In some instances, these protective coatings are formed from a single layer, in which case they will normally have a dry coating thickness of ≤100 microns, more typically ≤75 or even ≤50 microns. In other instances, they can be formed from multiple layers, as many as 30 or more, in which case they will normally have a dry coating thickness of ≤3,000 microns, more typically ≤2,000, ≤1,000, ≤750 or even ≤500 microns.

In accordance with this invention, this capability of liquid coating techniques is taken advantage of not only to provide tough, hard, durable, strongly adherent, edible, thin protective coatings on selected edible substrates but also to supply cannabis to the edible product ultimately produced in a predetermined, precisely-controlled amount.

For this purpose, any known liquid coating technique can be used including spraying, brushing, dipping, ultra-sonic coating, electrocoating, enrobing, curtain coating (i.e., passing the substrate under a falling curtain of a liquid coating material), pan coating techniques and the like. In addition, depending on the desired thickness of the protective glaze coating to be produced, these liquid coating techniques can be carried out a single time to produce a protective glaze coating formed from a single layer or multiple times to produce a protective glaze coating formed from multiple layers. In addition, both solution coating techniques in which the edible, film-forming resin is dissolved in the carrier liquid and dispersion coating techniques in which the edible, film-forming resin is dispersed in the carrier liquid can be used.

By using this approach, protective glaze coatings can be produced in accordance with this invention which have a dry coating thickness of as little as 50 microns or less, such as when a single layer coating is formed, and as much as 3,000 microns or more such as when a multiple layer coating is formed. Thus, dry coating thicknesses on the order of 50-3,000 microns, more typically 50-2,000 microns, 50-1,000 microns, 50-750 microns, 50-500 microns, 50-400 microns, 50-300 microns, 50-250 microns, 50-200 microns, 50-150 microns and even 50-100 microns can be formed. When formed from a single layer, these protective glaze coatings will normally have a dry coating thickness of ≤250 microns, ≤200 microns, ≤150 microns, ≤100 microns or even ≤50 microns. When formed from multiple layers, these protective glaze coatings will normally have a dry coating thickness of ≤3,000 microns, ≤2.000 microns, ≤1,000 microns, ≤750 microns, ≤500 microns, ≤450 microns, ≤400 microns, ≤350 microns, ≤300 microns, ≤250 microns, or even ≤200 microns.

Also, when the protective glaze coating of this invention is formed from multiple layers, the individual layers used can be selected to exhibit different characteristics and/or functionalities so that the protective glaze coating ultimately obtained exhibits a combination of properties that would be difficult or impossible to achieve in a protective glaze coating made from a single material. For example, a pre-coat made from shellac or a water soluble analogue can be applied to the substrate first by means of a water-based liquid glaze coating composition followed by applying second and subsequent coating layers by means of ethanol or other organic solvent-based liquid glaze coating compositions. This approach is common in chocolate, confectionery and pharmaceutical applications, where a water-based carbohydrate “polishing” agent is applied, then an ethanol-based shellac glaze is then applied, since it keeps the ethanol from absorbing into the chocolate/confectionary substrate.

A particularly interesting application method for use in this invention is pan coating techniques in which a liquid coating compositions is poured or sprayed onto a batch of substrates to be coated which are tumbling about in a rotating pan. Pan coating techniques are commonly used for providing confectioner's and pharmaceutical glaze coatings made from shellac, zein and/or ethyl cellulose on a variety of different types of hard and soft edible products such as pills, tablets, nuts, chocolates, chocolate covered nuts, hard sugar candies, gum balls, gummies, etc. It is a particularly advantageous application method for use in this invention, because the exact amount of coating composition that will be taken up by each individual substrate being coated can be precisely controlled. This feature, in combination with the advantageous rheological properties of the liquid glaze coating composition of this invention as described above, enable the amount of cannabis taken up by each individual substrate to be controlled with a very high degree of precision.

Incidentally, for the sake of clarification and defining terms, it should be understood that, for the purposes of this disclosure, “spraying” will be understood to refer to coating methods which are different from and do not include “pan coating techniques,” even if the coating liquid used in the pan coating technique is applied by spraying.

An important feature of liquid coating techniques, as mentioned above, is that they produce thin, essentially continuous protective resin coatings of a uniform and controlled thickness. For this reason, and because liquid coating techniques of this type are so well-developed, persons skilled in the art should have no difficulty in making tough, hard, durable, edible, strongly adherent, protective coatings having a desired thickness as described above and, in addition, also containing a desired concentration of cannabis as also described above by suitable selection of the various parameters involved in the coating process—such parameters including, but not limited to, the selection of the particular edible, film-forming resin, cannabis ingredient and carrier liquid used, the concentration of these ingredients in the liquid glaze coating composition and the particular coating technique used.

### Ethanol-Based Glaze Coating Compositions

In a particularly advantageous embodiment of this invention, the carrier liquid of the liquid glaze coating composition of this invention is a C₂-C₃ alcohol containing 1-3 hydroxyl groups, preferably ethanol, isopropanol or mixtures thereof. Ethanol is especially preferred, because it is readily available, inexpensive and approved for food use.

Ethanol extraction is an effective way of recovering cannabis compounds in general, and tetrahydrocannabinol (THC) and cannabidiol (CBD) in particular, from the cannabis sativa plant. This produces an ethanol-based liquid extract which can be directly used to formulate the ethanol-based cannabis-containing liquid glaze coating composition of this invention, with or without intermediate purifying and/or concentrating. Because both this liquid extract and this coating composition are based on ethanol, solubility, incompatibility and other problems that could arise if different solvents were used are avoided.

Moreover, even if low molecular weight alcohols other than ethanol are used, the advantages of solvent compatibility and cannabis protections are still achieved.

This embodiment of the invention, especially when ethanol is used as the carrier liquid, is especially useful when pan coating techniques are used for coating application. This is because pan coating techniques are widely used commercially for applying pharmaceutical and confectioner's glazes to a wide variety of different edible products. Therefore, this embodiment of the invention can be easily practiced on a wide variety of different edible substrates by current commercial producers with little or no modification to existing processes or equipment.

### Controlling Release Rate

In yet another embodiment of this invention, the rate at which the user metabolizes the cannabis ingredient supplied by the inventive cannabis-containing edible product can be controlled even more precisely by suitable selection of the properties and characteristics of its protective glaze coating.

In conventional cannabis containing foods such as illustrated in the above-noted US 2012/046351 to Hospodor, the cannabis ingredient is released for metabolization by the user essentially immediately upon ingestion by the user. This is because the food material which surrounds the cannabis ingredient in such products offers essentially no protection against its immediate extraction and metabolization by the user's bodily processes. For example, the sugar icings, jelly and butter coatings shown in Hospodor offer no protection against the immediate extraction, up-take and metabolism of the cannabis ingredients therein by the user's bodily processes.

In the inventive cannabis-containing edible product, however, the cannabis ingredient resists immediate metabolism for both physical and chemical reasons. Physically, the cannabis tends to remain embedded in the edible, film-forming resin, even after the inventive edible product is eaten, because the relatively tough nature of this film-forming resin retards physical separation of the cannabis ingredient from this material even as a result of chewing. Chemically, this embedding material is only slowly soluble in the acidic environment found in the stomach, and so chemical release of the cannabis ingredient from this embedding material is also slower. The result is that, when the inventive cannabis-containing edible product is used, release of the cannabis ingredient is slower and more sustained as compared with conventional cannabis containing foods in which no such protection is provided. This, in turn, results in metabolization of the cannabis ingredient by the user's body being inherently slower and more sustained over time as compared with conventional cannabis containing foods.

In yet another feature of this invention, the rate at which the inventive cannabis-containing food product releases cannabis for metabolism can be predetermined (i.e., controlled) by suitable selection of the properties and characteristics of the protective glaze coating that is formed. For example, the rate of metabolism can be increased by making this coating thinner, by selecting an edible, film-forming resin which dissolves faster in the stomach and/or by including a greater amount of a plasticizer in the coating. Similarly, the rate of metabolism can be decreased by making this coating thicker, by making this coating in multiple layers with different release characteristics, by selecting an edible, film-forming resin which dissolves slower in the stomach and/or by including a smaller amount of a plasticizer in the coating.

### Precision Supply of Cannabis Ingredient

As indicated above, the cannabis-containing protective glaze coating of this invention is similar, at least in some respects, to the pharmaceutical glazes, confectioner's glazes and other protective film coatings previously applied to different edible substrates in that it, too, is hard, tough, durable, thin, edible, and strongly adherent to the substrate on which it is coated.

In addition, like these earlier protective films, the protective glaze coating of this invention is also essentially tasteless, and preferably essentially colorless and essentially odorless, as well. In this context, “essentially tasteless” means that the protective glaze coating of this invention provides no more flavor or taste to the product being coated than a conventional unflavored film coating or glaze made from shellac, zein or ethyl cellulose. In the same way, “essentially colorless” and “essentially odorless” mean that the protective glaze coating of this invention provides no more color or odor to the product being coated than a conventional uncolored and unscented film coating or glaze made from shellac, zein or ethyl cellulose.

In addition, like these earlier protective films, the protective glaze coating of this invention preferably also has essentially no caloric value. In this context, “essentially no caloric value” will be understood to mean that, based on a given surface area of a food product being covered, the calorie content of this protective glaze coating is ≤50% of the calorie content of a typical sugar glaze coating applied to donuts, i.e., a sugar glaze coating made by applying a mixture of 2 cups powdered sugar, ¼ cup milk and a teaspoon of vanilla extract to the food product followed by drying. More typically, the calorie content of this protective glaze coating will be ≤60%, ≤70%, ≤80%, ≤90% or even ≤95% of the calorie content of this sugar glaze coating.

Accordingly, the protective glaze coatings of this invention normally provide substantially less calories to the food product being coated than a typical food glaze such as the icings, frostings, chocolate coatings, fruit toppings, jellies, butter coatings, and the like shown in the above-noted US 2012/046351 to Hospodor. Normally, this will be due to the fact that this protective glaze coating is so thin relative to a typical food glaze. So, for example, a typical food glaze such as an icing, frosting, chocolate coating, fruit topping or jelly will normally be ≥5000 microns thick, more typically ≥6,000, ≥7,500 or even ≥10,000 microns thick. In contrast, the protective glaze coating of this invention will normally have a dry coating thickness of ≤3,000 microns, more typically ≤2,000 microns, ≤1,000 microns, ≤750 microns or even ≤500 microns, when made from multiple layers and ≤500 microns, ≤400 microns, ≤300 microns, ≤200 microns, ≤100 microns, or even ≤50 microns when made from a single layer.

In addition to smaller thickness, another reason why the protective glaze coatings of this invention provide substantially less calories than a typical food glaze is that, at least in some embodiments, this protective glaze coating is made from an edible, film forming resins which has a very low calorie content—far lower than the materials used to form a conventional food glaze. For example, one of the edible, film-forming resins which can be used to form the protective glaze coating of this invention, shellac, is essentially calorie-free. Protective glaze coatings made from shellac will have “essentially no caloric value” at least because the calorie content of shellac is low. Protective glaze coatings which are made from edible, film forming resins having a higher caloric content such as zein and ethyl cellulose, for example, normally will have “essentially no caloric value” primarily because they are so thin.

In any event, in addition to being essentially tasteless, the dry protective glaze coatings of this invention normally differ from food glazes of the type shown in the above-noted Hospodor disclosure for the additional reason that they have essentially no caloric value as well.

In addition to these difference, in some embodiments, the protective glaze coatings of this invention provide additional beneficial functional features not found in food glazes such as those shown in the Hospodor disclosure. Examples of these additional functional features include toughness, durability, hardness, oxygen-impermeability, water vapor-impermeability and the like. For example, protective glaze coatings made from shellac, in addition to being essentially tasteless, essentially odorless, essentially colorless, and essentially calorie free are also hard, tough, durable and exhibit excellent barrier properties against the transmission of atmospheric oxygen and water vapor as well.

Thus, in some embodiments, the protective glaze coatings of this invention will also be one or more of tough, durable, oxygen-impermeable and water vapor-impermeable. In this regard, for the purposes of this disclosure, it will be understood that a protective glaze coating will be considered to be “hard” or exhibit “hardness” if it exhibits a hardness level of at least F when tested by the Pencil Hardness Test of ASTM Method D3363. Preferred protective glaze coatings exhibit pencil hardness levels of at least HB, B, 2B or even 3B when measured by this test.

Similarly, a protective glaze coating of this invention will be understood to be “oxygen-impermeable” if its permeability to atmospheric oxygen at STP (standard temperature and pressure) is no more than double that of refined beach shellac. Preferred protective glaze coatings have oxygen permeabilities at STP which are ≤175%, ≤150%, ≤125%, or even ≤100% of that of refined bleached shellac. Thus, lower oxygen permeabilities are better, with oxygen permeabilities twice as much as that of refined beach shellac or less being “oxygen-impermeable” within the meaning of this disclosure.

In the same way, a protective glaze coating of this invention will be understood to be “water vapor-impermeable” if its permeability to atmospheric water vapor at STP is no more than double that of refined beach shellac. Preferred protective glaze coatings have water vapor-permeabilities at STP which are ≤175%, ≤150%, ≤125%, or even ≤100% of that of refined bleached shellac. Thus, lower water vapor-permeabilities are better, with water vapor-permeabilities twice as much as that of refined beach shellac or less being “water vapor-impermeable” within the meaning of this disclosure.

Accordingly, it will be understood that the protective glaze coating of this invention differs from food glazes in general, and the food glazes mentioned in the above-noted US 2012/046351 to Hospodor in particular in that food glazes are a type of coating or topping which is intentionally formulated to alter (generally to improve) the flavor of the food product to which they are applied. They are not intentionally formulated to provide any meaningful protection to the food product being coated and so they generally contain no ingredient such as the shellac, zein and/or ethyl cellulose found in confectioner's and pharmaceutical glazes which can provide this protection. Icings, frostings, chocolate coatings, fruit toppings, jellies, butter coatings, etc. are good examples of these food glazes, since each contains an ingredient such as sugar, chocolate, fruit or butter which significantly changes the taste and caloric content of the food substrate being coated but do not contain any ingredient which is capable of providing meaningful protection to this food product. Thus, it will be appreciated that “food” in “food glaze” means only that the glaze itself has significant food value in terms of taste, caloric content or both. It does not mean that, regardless of its inherent food value, the glaze has been especially adapted for applying to foods.

In any event, the protective glaze coating of this invention is not a “food glaze,” as that term is commonly understood, because it is essentially tasteless and preferably is very thin as well as having essentially no caloric content as well. Normally, it is also hard, oxygen-impermeable and water vapor-impermeable as well.

Because of these differences, it is far easier to precisely meter or control the exact amount of cannabis that is supplied to the user as compared to when conventional food glazes are used for this purpose. One reason for this is because the liquid glaze coating composition of this invention, being similar to the coating compositions used to form conventional confectioner's glazes and pharmaceutical glazes in term of physical properties, is highly liquid in form rather than being highly viscous semi-solids. To this end, the viscosity of the liquid glaze coating composition of this invention will normally be ≤500 cps, more typically ≤400 cps, ≤300 cps, ≤200 cps, ≤100 cps ≤75 cps, ≤50 cps, ≤25 cps, ≤10 cps, or even ≤5 cps. This relatively low viscosity makes it inherently easier to meter the exact amount of liquid glaze coating composition applied due to its easy flow characteristics. In contrast, many food glazes such as the jellies, icings, frostings and other food toppings shown in the above-noted US 2012/046351 to Hospodor do not flow at all, while those that do flow do so only very slowly due to viscosities which can easily be ≥1,000 cps, ≥5,000 cps, ≥10,000 cps, ≥50,000 cps, or even ≥100,000 cps. Such high viscosity materials are inherently more difficult to meter accurately due to their poor, or non-existent, flow characteristics.

Another reason why the exact amount of cannabis that is supplied to the user can be precisely metered and controlled in accordance with this invention is that it is easier to achieve a homogeneous distribution of the cannabis ingredient in the liquid glaze coating composition of this invention as compared with conventional food glazes such as those shown in the above-noted US 2012/046351 to Hospodor. due to its chemical composition and viscosity.

The liquid glaze coating composition of this invention has a relatively low viscosity, while a conventional food glaze such as the jellies, icings, frostings and the like shown in the above-noted US 2012/046351 to Hospodor have high viscosities, if they flow at all. As well understood in the art, it is inherently more difficult to achieve a homogeneous distribution of ingredients in a mixture having a higher viscosity as opposed to a lower viscosity due to the greater amount of force and energy required to achieve the same amount of mixing. As a result, it is inherently more difficult to insure that the coatings applied to successive edible substrates contain exactly the same concentration of cannabis ingredient, from one edible substrate to the next, when higher viscosity coating compositions are used relative to lower viscosity coating compositions due to concerns relating to the potential non-uniform distribution of cannabis ingredient in these coatings. Because the liquid glaze coating compositions of this invention have far lower viscosities than conventional food glazes, it is far more likely that the concentration of cannabis ingredient in each dose of liquid glaze coating composition deposited on successive edible substrates is the same and hence that the amount of cannabis ingredient deposited on each successive edible substrate is the same.

Still another reason why the exact amount of cannabis that is supplied to the user can be precisely metered and controlled in accordance with this invention is that liquid coating techniques are used to form the cannabis-containing protective glaze coating of this invention. As appreciated in the art, this type of coating process inherently produces a film of a particular uniform thickness which, in turn, enables the total amount of coating applied and hence the total amount of cannabis ingredient supplied to be precisely controlled.

In contrast, typical food glazes such as the jellies, icings, frostings and other food toppings shown in the above-noted US 2012/046351 to Hospodor are generally applied to substrates in the form of globs and blobs which must be mechanically spread to produce the coating. Obviously, it is much more difficult to control the thickness and uniformity of a coating applied in this way as compared with a thin resin film coating formed by liquid coating techniques.

Yet another reason why the exact amount of cannabis that is supplied to the user can be precisely metered and controlled in accordance with this invention is that the protective glaze coating that is formed creates a barrier which effectively protects the applied cannabis ingredient against loss and/or degradation during storage, handling and use. This occurs not only because the excellent barrier properties of the film-forming resin in this protective glaze coating protect the cannabis ingredient from chemical attack by atmospheric oxygen and water vapor but also because the inherently tough nature of this film-forming resin also protects the cannabis ingredient from physical degradation as well.

In contrast, the ingredients which form the vast majority of typical food glazes such as the jellies, icings, frostings and other food toppings shown in the above-noted US 2012/046351 to Hospodor (e.g., sugar, butter, gelatinized fruits, etc.) exhibit no such barrier property or toughness. As a result, the food glaze coatings described there can be easily damaged, and hence the cannabis ingredient therein compromised, during storage, shipment and use.

Although only a few embodiments of this invention have been described above, it should be appreciated that many modifications can be made without departing from the spirit and scope of the invention. All such modifications are intended to be included within the scope of this invention, which is to be limited only by the following claims.

1. A cannabis-containing edible product comprising
an edible substrate and
a predetermined amount of a protective glaze coating on at least a portion of a surface of the edible substrate,
wherein the protective glaze coating contains cannabis in a predetermined concentration such that the cannabis-containing edible product contains a predetermined amount of cannabis,
wherein the protective glaze coating is essentially tasteless and comprises an edible, film-forming resin.

2. The cannabis-containing edible product of claim 1, wherein the protective glaze coating comprises an essentially continuous layer of the edible, film-forming resin.

3. The cannabis-containing edible product of claim 1, wherein the protective glaze coating exhibits one or more of the following additional properties:
a hardness of F or greater, as determined by the Pencil Hardness Test of ASTM D3363,
an oxygen-permeability of no more than double that of refined beach shellac, and
a water vapor-permeability of no more than double that of refined beach shellac.

4. The cannabis-containing edible product of claim 1, wherein the protective glaze coating comprises a single layer having a dry coating thickness of less than or equal to 200 microns.

5. The cannabis-containing edible product of claim 4, wherein the single layer has a dry coating thickness of less than or equal to 100 microns.

6. The cannabis-containing edible product of claim 1, wherein the protective glaze coating comprises multiple layers each having a dry coating thickness of less than or equal to 200 microns, and further wherein the total thickness of the protective layer is less than or equal to 3000 microns.

7. The cannabis-containing edible product of claim 1, wherein the protective glaze coating comprises multiple layers each having a dry coating thickness of less than or equal to 100 microns, and further wherein the total thickness of the protective layer is less than or equal to 1000 microns.

8. The cannabis-containing edible product of claim 1, wherein the protective glaze coating comprises a pre-coat which has been applied to the edible substrate by means of a water-based protective glaze coating composition and one or more subsequent coating layers which have been applied by means of an organic solvent-based protective glaze coating composition.

9. The cannabis-containing edible product of claim 1, wherein the protective glaze coating is formulated to release cannabis at a predetermined rate.

10. The cannabis-containing edible product of claim 9, wherein the protective glaze coating is formulated to release cannabis at a predetermined rate by making this coating in multiple layers with different release characteristics.

11. The cannabis-containing edible product of claim 1, wherein the protective glaze coating has essentially no caloric content.

12. The cannabis-containing edible product of claim 1, wherein the edible, film-forming resin is ethanol-soluble.

13. The cannabis-containing edible product of claim 1, wherein the edible, film-forming resin comprises shellac, zein, ethyl cellulose or a mixture thereof.

14. The cannabis-containing edible product of claim 1, wherein the edible substrate is a foodstuff.

15. The cannabis-containing edible product of claim 14, wherein the foodstuff is a bakery product.

16. The cannabis-containing edible product of claim 1, wherein the protective glaze coating is applied to the surface of the edible substrate by pan coating techniques.

17. The cannabis-containing edible product of claim 1, wherein the cannabis comprises tetrahydrocannabinol (THC), cannabidiol (CBD) or a mixture thereof.

18. A process for producing a cannabis-containing edible product comprising an edible substrate, the process comprising:
applying a predetermined amount of a liquid glaze coating composition onto at least a portion of a surface of the edible substrate; and
drying the edible substrate and liquid glaze coating composition to produce a protective glaze coating,
wherein the liquid glaze coating composition comprises an edible, film-forming resin, a carrier liquid and cannabis in a predetermined concentration such that the cannabis-containing edible product contains a predetermined amount of cannabis, wherein the liquid glaze coating composition is formulated so that the protective glaze coating obtained is essentially tasteless and comprises an essentially continuous layer of an edible, film-forming resin.

19. The process of claim 18, wherein the liquid glaze coating composition is formulated so that the protective glaze coating obtained exhibits one or more of the following additional properties:
a hardness of F or greater, as determined by the Pencil Hardness Test of ASTM D3363,
an oxygen-permeability of no more than double that of refined beach shellac, and
a water vapor-permeability of no more than double that of refined beach shellac.

20. The process of claim 18, wherein the protective glaze coating comprises a single layer having a dry coating thickness of less than or equal to 100 microns.

21. The process of claim 18, wherein the protective glaze coating comprises multiple layers each having a dry coating thickness of less than or equal to 100 microns, and further wherein the total thickness of the protective layer is less than or equal to 1000 microns.

22. The process of claim 18, wherein the liquid glaze coating composition is formulated so that the protective glaze coating obtained has essentially no caloric value.

23. The process of claim 18, wherein the edible, film-forming resin is ethanol-soluble.

24. The process of claim 18, wherein the edible, film-forming resin comprises shellac, zein or a mixture thereof.

25. The process of claim 18, wherein the carrier liquid is water, one or more C1-C5 oxygen-containing organic solvents, or mixtures thereof.

26. The process of claim 18, wherein the liquid glaze coating composition is applied to the edible substrate by pan coating techniques.

27. The process of claim 25, wherein the organic solvent in the liquid glaze coating composition is a C2-C3 alcohol or mixture of alcohols containing 1-3 hydroxyl groups, wherein the cannabis in the liquid glaze coating composition is a compound or a mixture of compounds which is supplied to the liquid coating composition by means of an extract of the cannabis sativa plant, and further wherein the extract also comprises an organic solvent which is a C2-C3 alcohol or mixture of alcohols containing 1-3 hydroxyl groups.

28. The process of claim 18, wherein the cannabis comprises tetrahydrocannabinol (THC), cannabidiol (CBO) or a mixture thereof.
